(19) United States
(12) Patent Application Publication
(10) Pub. No.: US 2004/0214845 A1
(43) Pub. Date: Oct. 28, 2004

(54) Barbiturates as integrin antagonists and their use for treating inflammatory diseases

(75) Inventors: Marc Bazin, Arcueil (FR); Geraldine C. Harriman, Charlestown, RI (US); Cyrille Kuhn, Paris (FR); Jay R. Luly, Wellesley, MA (US)

Correspondence Address: MILLENNIUM PHARMACEUTICALS, INC., 40 Landsdowne Street, CAMBRIDGE, MA, 02139, US

(73) Assignee: Millennium Pharmaceuticals, Inc.

(21) Appl. No.: 10/848,718

(22) Filed: May 19, 2004

Related U.S. Application Data
(63) Continuation of application No. 10/080,864, filed on Feb. 22, 2002.
(60) Provisional application No. 60/270,503, filed on Feb. 22, 2001.

(51) Int. Cl. A61K 31/515

(52) U.S. Cl. 514/270

(57) ABSTRACT

The invention relates to a pharmaceutical composition comprising a compound of the formula (I):

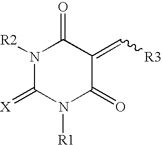

in which X is O or S; R1 is hydrogen or —(CH₂)ₙ—Ar1; R2 is hydrogen or has the same meaning as R1; R3 is Ar2 or —(CH═CH)—Ar2 where Ar2 has the same meaning as Ar1; together with a pharmaceutically acceptable carrier.

The invention also relates to a method of treating an individual suffering from a disease associated with leukocyte infiltration of tissues expressing the molecule MAdCAM-1, comprising administering a therapeutically effective amount of a compound of formula (I).

The invention also relates to a method of inhibiting the binding of a cell expressing a ligand for MAdCAM-1 on its surface to MAdCAM-1 or a portion thereof.

The invention further relates to a method of preparing a pharmaceutical composition comprising a compound of the formula (I).

### RELATED APPLICATIONS

This application claims priority to U.S. Provisional Application Serial No. 60/270,503, filed Feb. 22, 2001, entitled “Barbiturates as Integrin Antagonists and Their Use for Treating Inflammatory Diseases.” The entire contents of this application are hereby incorporated herein by reference.

### FIELD OF THE INVENTION

The present invention relates to barbiturates as integrin antagonists, and their use for treating notably inflammatory diseases.

### BACKGROUND OF THE INVENTION

Lymphocyte homing from the circulation to the lymphoid tissues, and migration to sites of inflammation, is regulated by interaction with receptors expressed in postcapillary venules, including high endothelial venules found in secondary lymphoid tissues (e.g. mesenteric lymph nodes, Peyer's patches) (Bevilacqua, M. P., Annu. Rev. Immunol., 11:767-804 (1993); Butcher, E. C., Cell, 67: 1033-1036 (1991); Picker, L. J., et al., Annu. Rev. Immunol., 10:561-591 (1992); and Springer, T A., Cell, 76: 301-314 (1994)). These interactions are tissue specific in nature.

Inflammation (e.g. chronic inflammation) is characterized by infiltration of the affected tissue by leukocytes, such as lymphocytes, lymphoblasts, and mononuclear phagocytes. The remarkable selectivity by which leukocytes preferentially migrate to particular tissues during both normal circulation and inflammation results from a series of adhesive and activating events involving multiple receptor-ligand interactions. See Butcher and others (Butcher, E. C., Cell, 67: 1033-1036 (1991); vonAdrian, U. H., et al., Proc. Natl. Acad. Sci. USA, 88:7538 (1991); Mayadas, T. N., et al., Cell, 74:541 (1993); (Springer, T. A., Cell, 76:301 (1994)).

As an initial step, there is a transient, rolling, interaction between leukocytes and the endothelium, which results from the interaction of selectins (and by α4 integrins in some instances) with their carbohydrate ligands. This interaction, which is characterized by rolling in the direction of flow, can be assessed by known methods (Lawrence, M B. and T. A. Springer, Cell, 65:859 (1991); WO-A-9221746, Springer et al., (Dec. 10, 1992). This is followed by activation events mediated by chemoattractants such as chemokines and their receptors, which cause activation of integrin adhesiveness, and influence the direction of migration of leukocytes through vascular walls. Such secondary signals in turn trigger:

i) the firm adhesion of leukocytes to the endothelium via interactions between leukocyte integrins and their endothelial ligands (Ig-like proteins), and

ii) subsequent transendothelial migration from the circulation across the vascular endothelium via interactions between leukocyte integrins and their extracellular matrix ligands (fibronectin).

In secondary lymphoid tissues, such as Peyer's patches and lymph nodes (e.g. peripheral lymph nodes), leukocyte trafficking and homing is regulated by interactions of homing receptors on the surface of leukocytes with endothelial cells lining the post-capillary venules, notably the high endothelial venules (Gowans, J. L. and E. J. Knight, Proc. R. Soc. Lond., 159:257 (1964)). Receptors terned Vascular Cell Addressing Molecules (VCAM), which are present on the surface of endothelial cells, regulate the migration and subsequent extravasation of lymphocyte subsets. VCAMs show restricted patterns of expression and this tissue-specific expression makes an important contribution to the specificity of leukocyte trafficking (Picker, L J. and E. C. Butcher, Annu. Rev. Immunol., 10:561-591 (1992); Berg, E L., et al., Cellular and molecular mechanisms of inflammation, 2:111 (1991); Butcher, E. C., Cell, 67:1033-1036 (1991)).

Mucosal vascular addressing MAdCAM-1 (Mucosal Addressing Cell Adhesion Molecule-1) is an immunoglobulin superfamily adhesion receptor for lymphocytes, which is distinct from VCAM-1 and ICAM-1 (Intercellular Adhesion Molecule-1).

MAdCAM-1 was identified in the mouse as a ˜60 kD glycoprotein that is selectively expressed at sites of lymphocyte extravasation. In particular MAdCAM-1 expression is reported in vascular endothelial cells of mucosal tissues, including gut-associated tissues or lymph of the small and large intestine, and the lactating mammary gland, but not in peripheral lymph nodes. MAdCAM-1 is involved in lymphocyte binding to Peyer's patches. (Streeter, P R., et al., Nature, 331:41-46 (1988); Nakache, M., et al., Nature, 337: 179-181 (1989); Picker, L. J., et al., Annu. Rev. Immunol., 10:561-591 (1992); Briskin, M. J., et al., Nature, 363:461 (1993); Berg, E. L., et al., Nature, 366:695-698 (1993); Berlin, C., et al., Cell, 74:185-195 (1993)). MAdCAM-1 can be induced in vitro by proinflammatory stimuli (Sikorski, E. E., et al., J. Immunol., 151:5239-5250 (1993)).

cDNA clones encoding murine and primate (e.g. human) MAdCAM-1 have been isolated and sequenced (Briskin, M. J. et al., Nature, 363:461-464 (1993); Briskin et al., WO-A-9624673, published Aug. 15, 1996; and Briskin, M. J. et al., U.S. Ser. No. 08/523,004, filed Sep. 1, 1995, the priority thereof, the teachings of each of which are incorporated herein by reference in their entirety).

MAdCAM-1 specifically binds the lymphocyte integrin α4β7 (also referred to as LPAM-1 (mouse), α4βp (mouse)), which is a lymphocyte homing receptor involved in homing to Peyer's patches (Berlin, C., et al., Cell, 80:413-422 (1994); Berlin, C., et al., Cell, 74:185-195 (1993); and Erle, D. J., et al., J. Immunol., 153: 517-528 (1994)).

In contrast to VCAM-1 and fibronectin, which interact with both α4β1 and αβ7 (Berlin, C., et al., Cell, 74: 185-195 (1993); Strauch, U. S., et al., Int. Immunol., 6:263 (1994)), MAdCAM-1 is a selective receptor for α4β7.

Inflammatory bowel disease (IBD), such as ulcerative colitis and Crohn's disease, for example, can be a debilitating and progressive disease involving inflammation of the gastrointestinal tract. Affecting an estimated two million people in the United States alone, symptoms include abdominal pain, cramping, diarrhea and rectal bleeding. IBD treatments have included anti-inflammatory drugs (such as corticosteroids and sulfasalazine), immunosuppressive drugs (such as 6-mercaptopurine, cyclosporine and azathioprine) and surgery such as colectomy (Podolsky, New Engl. J. Med., 325:928-937 (1991) and Podolsky, New Engl. J. Med., 325: 1008-1016 (1991). Such treatments are unwieldy and often associated with serious side effects.

Thus there is a need for inhibitors of MAdCAM-1 function to provide new therapies useful in the treatment of IBD and other inflammatory diseases involving leukocyte infiltration of the gastrointestinal tract or other mucosal tissues. There is a need for new therapies for treating, e.g. inflammation, immune disorders, asthma, chronic obstructive pulmonary disease (COPD), multiple sclerosis, and other inflammatory disorders.

### SUMMARY OF THE INVENTION

The invention relates to pharmaceutical compositions comprising the barbiturates of formula (I)

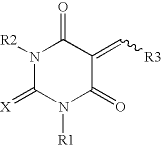

in which:

X is O or S;

R1 is H or —(CH₂)ₙ—Ar1, in which

n is 0, 1 or 2;

Ar1 is an aryl or heteroaryl group, optionally substituted;

R2 is hydrogen or has independently the same meaning as R1;

R3 is Ar2 or —(CH═CH)—Ar2 where Ar2 has independently the same meaning as Ar1;

as well as the pharmaceutically acceptable derivatives thereof,

together with a pharmaceutically acceptable carrier.

The barbiturates of the invention specifically bind to α4β7 integrins thereby inhibiting adhesion to MAdCAM-1 of those leukocytes that express α4β7 on their cell surface.

Also provided are methods of inhibiting the interaction of a cell bearing a ligand of MAdCAM-1, including α4β7 integrins, with MAdCAM-1 or a portion thereof (e.g. the extracellular domain), comprising contacting the cell with a barbiturate of the present invention.

In one embodiment, the invention relates to a method of inhibiting the MAdCAM-mediated interaction of a first cell bearing an α4β7 integrin with MAdCAM, for example with a second cell bearing MAdCAM, comprising contacting the first cell with a barbiturate of the present invention.

In another embodiment, the invention relates to a method of treating an individual suffering from a disease associated with leukocyte recruitment to tissues expressing the molecule MAdCAM-1 (e.g. on endothelial cells). Said method comprises administering a therapeutically effective amount of an integrin antagonist of formula (I).

The invention also relates to a method of inhibiting the binding of a cell (such as a leukocyte) expressing on its surface a ligand for MadCAM-1 (e.g. the integrin α4β7) to MAdCAM-1, and thus to endothelial cells expressing MAdCAM-1 on their surface. The method comprises contacting the former cell with an effective amount of an integrin antagonist of formula (I).

The invention also relates to a process for preparing the above compositions.

### DETAILED DESCRIPTION OF THE INVENTION

The invention is directed to pharmaceutical compositions comprising the barbiturate of the following formula (I)

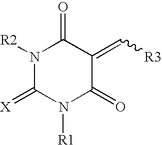

in which:

X is O or S;

R1 is H or —(CH₂)ₙ—Ar1, in which

n is 0, 1 or 2;

Ar1 is an aryl or heteroaryl group, optionally being substituted with one to three groups chosen independently from:

halogen,

cyano,

hydroxy,

carboxyl,

C₁-C₄ alkyl, optionally substituted with halo, cyano, hydroxy, carboxy,

C₁-C₄ haloalkyl,

C₁-C₄ alkoxy,

NR′R″ where R′ and R″ independently are hydrogen or a C₁-C₄ alkyl;

R2 is hydrogen or has independently the same meaning as R1;

R3 is Ar2 or —(CH═CH)—Ar2 where Ar2 has independently the same meaning as Ar1;

as well as the pharmaceutically acceptable derivatives thereof,

together with a pharmaceutically acceptable carrier;

In the following and in the foregoing text:

aryl is understood to refer to phenyl or naphthyl or tetrahydronaphthyl;

heteroaryl is understood to refer to a non-saturated monocycle or polycycle containing at least one heteroatom such as nitrogen, oxygen, or sulfur and, preferably, five- to ten-membered heteromonocycles or heterobicycles containing from 1 to 4 nitrogen or sulfur or oxygen atoms, most preferably a heterobicycle containing one nitrogen atom; suitable heterocyles include notably monocycles such as 2- and 3-furanyl, 2- and 3-thienyl, 2-pyridyl, 2- and 3-pyranyl, as well as fulsed rings such as 2- and 3-benzothienyl, 2- and 3-benzofuranyl, 2-indolyl, 2- and 3-quinolinyl, acridinyl and 9-thioxantanyl.

halogen is understood to refer to fluorine, chlorine, bromine or iodine;

as regards radicals comprising an alkyl sequence, lower is understood to mean that the alkyl is linear or branched and contains from one to four carbon atoms, or alternatively represents the cyclopropylmethyl radical;

haloalkyl is understood to refer to a mono-, di- or trihaloalkyl.

The compounds utilized in the invention include solvates, hydrates, pharmaceutically acceptable salts, and polymorphs (different crystalline lattice descriptors) of the compound of formula (I), which are pharmaceutically acceptable derivatives thereof.

The expression pharmaceutically acceptable salt of a compound of formula (I) having a basic part should be understood to refer to the addition salts of the compounds of formula (I) which may be formed from non-toxic inorganic or organic acids such as, for example, hydrobromic, hydrochloric, sulfuric, phosphoric, nitric, acetic, succinic, tartaric, citric, maleic, hydroxymaleic, benzoic, fumaric, toluenesulfonic and isethionic acid salts, and the like. The various quaternary ammonium salts of the derivatives (I) are also included in this category of compounds of the invention. In addition, the expression pharmaceutically acceptable salt of a compound of formula (I) having an acidic part is understood to refer to the usual salts of the compounds of formula (I) which may be formed from non-toxic inorganic or organic bases such as, for example, the hydroxides of alkali metals and alkaline-earth metals (sodium, potassium, magnesium and calcium), amines (dibenzylethylenediamine, trimethylamine, piperidine, pyrrolidine, benzylamine and the like) or alternatively quaternary anunonium hydroxides such as tetramethylammonium hydroxide(See also Berge S. M. et al. (1997) J. Pharm. Sci. 66: 1-19, which is incorporated herein by reference.). Use of a prodrug of a compound of formula (I), such as would occur to one skilled in the art (see Bundgaard, et al., Acta Pharm. Suec., 1987; 24: 233-246), is also contemplated.

One preferred class of the barbiturates of formula (I) in which R1 is —(CH₂)ₙ—Ar1, n and Ar1 being such as previously defined, and R2 has independently the same meaning as R1.

Another preferred class of the barbiturates of formula (I) in which R 1 is —(CH₂)ₙ—Ar1, n and Ar1 being such as previously defined, and R2 is hydrogen.

One preferred class of the barbiturates of formula (I) is the class in which R2 is hydrogen.

Another preferred class of the barbiturates of formula (I) is the class in which X is O.

Another preferred class of the barbiturates of formula (I) is the class in which R1 is H or —(CH₂)ₙ—Ar1, in which

n is 0 or 1;

Ar1 is an aryl or heteroaryl group, optionally being substituted with one or two groups chosen independently from halogen, C₁-C₄ alkyl and C₁-C₄ alkoxy.

Another preferred class of the barbiturates of formula (I) is the class in which R3 is Ar2 or —(CH═CH)—Ar2 where Ar2 is an aryl or heteroaryl group, optionally being substituted with one C₁-C₄ alkyl group.

One particularly preferred class is the class in which R1 is selected from the following substituents:

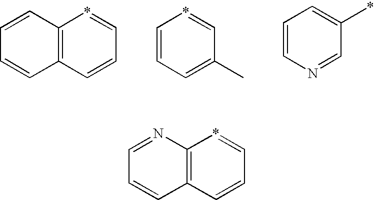

wherein the * indicates the site where R1 is linked to the rest of (I).

Another particularly preferred class is the class in which R3 is selected from the following substituents:

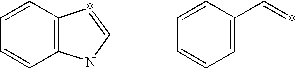

wherein the * indicates the site where R1 is linked to the rest of (I).

More particularly, the following compounds (I):

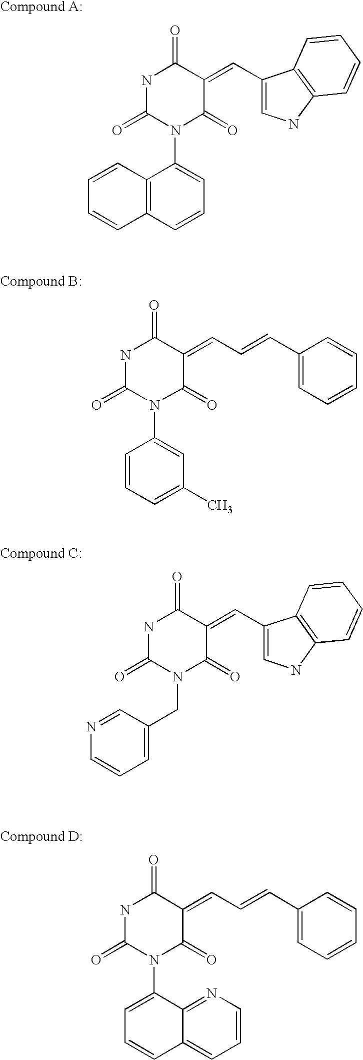

are preferred.

Preparation Process.

The compounds of this invention can be synthesized according to four general procedures of synthesis, procedures A to D, utilizing the methodology hereinafter described; types of reaction are known to a person skilled in the art. See for barbituric ring formation: Levina R. Y. and Velichko, Russian Chemical Review, 1960, 437-459. See for synthesis of 5-arylidene-barbiturates: Jones G., Organic Reactions, 1967, 15, 204-599.

The starting compounds (urea or thiourea; aldehyde) are either commercially available or can be synthesized using known procedures. (For example, the (thio)urea will be obtained starting from respectively KSCN or KOCN). The aldehyde may alternatively be obtained by reduction of the corresponding ester.

The process for making the compounds of the invention comprises two main steps:

The first step is the barbiturate ring formation (with a malonic derivative in which L is a leaving group such as Cl or OEt) while the second step is the 5-arylidene-1-N-substituted barbiturate synthesis. The two-step synthesis can be carried out with or without intermediate purification and isolation.

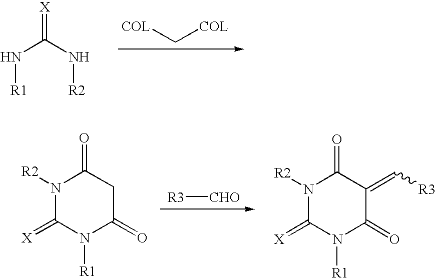

More specifically, these two steps can be carried out according to two preferred routes, each time:

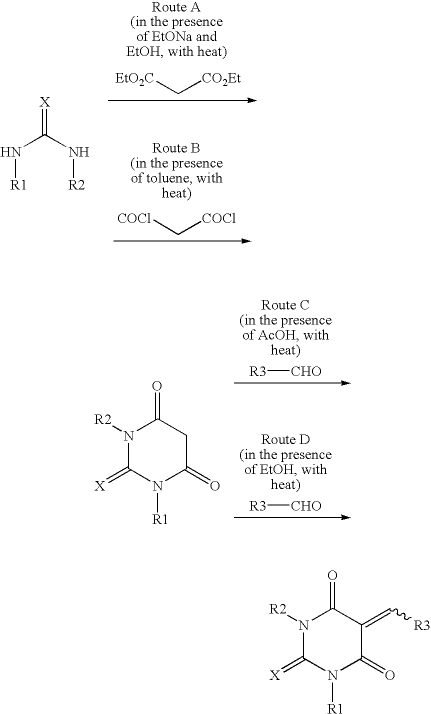

Routes A and B can be combined with C and D, according to any combination A+C, A+D, B+C, B+D, where the + indicates that an intermediate purification and separation occurred.

Altematively, the steps can be carried out in one pot, with a mere concentration after the first step. Again, any order combination can be followed: AC, AD, BC or BD; the lack of a “+” in the abbreviation indicates that the second step is carried out on the crude product obtained in the first step.

The solvent, reaction time, temperature, catalyst if any, can be varied, as the skilled man will appreciate.

Routes AC and BD are preferred.

Pharmaceutical Compositions.

The products of the invention are administered in the form of compositions, which are appropriate for the nature, and severity of the complaint to be treated. The daily dose in humans is usually between 2 mg and 1 g of product, which may be taken in one or more individual doses. The compositions are prepared in forms which are compatible with the intended route of administration, such as, for example, tablets, coated tablets, capsules, mouthwashes, aerosols, powders for inhalation, suppositories, enemas, foams (such as rectal foams) gels or suspensions. These compositions are prepared by methods which are familiar to those skilled in the art. They comprise from 0.5 to 60% by weight of active principle (compound of formula I) and 40 to 99.5% by weight of a pharmaceutical vehicle or carrier which is appropriate and compatible with the active principle and the physical form of the intended composition.

Solid form preparations include powders, tablets, dispersible granules, capsules, cachets, and suppositories. A solid carrier can be one or more substances which may also act as diluents, flavoring agents, solubilizers, lubricants, suspending agents, binders, or tablet disintegrating agents; it can also be an encapsulating material. In powders, the carrier is a finely divided solid, which is in a mixture with the finely divided active component. In tablets, the active component is mixed with the carrier having the necessary binding properties in suitable proportions and compacted in the shape and size desired. For preparing suppository preparations, a low-melting wax such as a mixture of fatty acid glycerides and cocoa butter is first melted, and the active ingredient is dispersed therein by, for example, stirring. The molten homogeneous mixture is then poured into convenient sized molds and allowed to cool and solidify. The powders, tablets, cachets or encapsulated forms for capsules preferably contain 5% to about 70% of the active component. Suitable carriers are magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Tablets, powders, cachets, and capsules can be used as solid dosage forms suitable for oral administration. The drug may be delivered as a spray (either in a pressurized container fitted with an appropriate valve or in a non-pressurized container fitted with a metering valve).

Liquid form preparations include solutions, suspensions, and emulsions.

Sterile water or water-propylene glycol solutions of the active compounds may be mentioned as an example of liquid preparations suitable for parenteral administration. Liquid preparations can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methylcellulose, sodium carboxymethyl cellulose, and other suspending agents known in the art of pharmaceutical formulation.

Enemas are obtained according to known procedures to prepare solutions adapted for rectal administration. Foams are prepared according to known methods (these foams can notably be similar to those used to administer a drug such as 5-ASA for treating recto-colitis).

Preferably the pharmaceutical preparation is in unit dosage form. In such form, the preparation is divided into unit doses containing appropriate quantities of the drug. The unit dosage form can be a packaged preparation, the package containing discrete quantities of the preparation, for example, packaged tablets, capsules, and powders in vials or ampoules. The unit dosage form can also be a capsule, cachet, or tablet itself, or it can be the appropriate number of any of these packaged forms.

The dosage form will generally be from about 1 mg to about 1000 mg per day. Preferred doses will be from about 2 mg to about 500 mg per day (for an adult of 70 kg).

Methods of Using

The barbiturates of the invention are inhibitors of the binding of α4β7 to the receptor MAdCAM-1. Since the barbiturates are α4β7 integrins antagonists, they are therefore useful in the treatment of diseases such as inflamrnation, immune disorders, asthma, chronic obstructive pulmonary disease (COPD), multiple sclerosis (MS), inflammatory disorders, and notably inflammatory bowel disease (IBD). They have the potential for fewer side effects in other tissues where adhesion is mediated by α4β1 integrin, for example.

The barbiturates of the invention are also useful in diagnostic and research applications. For example, the barbiturates can be used as immunogens (e.g. when immobilized on a suitable carrier) to induce the formation of antibodies which selectively bind MAdCAM-1 or a portion thereof. These antibodies can in turn be used to identify cells expressing MAdCAM-1 on their surface or detect MAdCAM-1 in a sample.

The invention also provides a method for the treatment of inflammatory diseases (such as depicted above) comprising administering to a human in need thereof an effective amount of a barbiturate of formula (I).

### EXAMPLES

The following examples illustrate the invention without limiting it.

In the examples, the following HPLC and Mass Spectral analyses are followed. All of the final compounds were analyzed by reversed-phase HPLC using a Nucleosil C18, 150×4.6 mm id column or a Kromasil C18 50×2.1 mm id column, 5 μm. Elution was carried out with a linear gradient of 0.1% TFA in CH₃CN/Water (5% CH₃CN/95% Water, v/v) to 0.1% TFA in CH₃CN/Water (95% CH₃CN/5% Water, v/v) over a 30- or 15-minute period of time with flow rate of 1 or 0.25 ml/min. The purity of the samples was determined: they were essentially found to contain one component. This was confirmed by Atmospheric Pressure Chemical Ionization positive and negative mode (APCI+/−) or Electro Spray Ionization (ESI+/−) mass spectral analysis, cone voltage 20 V (Platform LC, Micromass).

### Route A

To a urea (0.5 mmol) is added a 1 M ethanolic solution of sodium ethoxide (3 equivalents) and diethyl malonate (1.1 equivalent). The mixture is warmed to 75° C. for 24 h. After cooling the solution is neutralized with concentrated HCl (1 equivalent). The precipitated solid is filtered, washed with ethanol, and dried under vacuum.

### Route B

To a urea (0.5 mmol) is added malonyl dichloride (1.1 equivalent) in a solution in toluene. The mixture is warmed at 105° C. for 4 h. The precipitated solid is filtered and dried under vacuum.

### Route C

To a suspension of 1-N-substituted-barbiturate (0.5 mmol) in acetic acid (3 ml) is added an aldehyde (1 equivalent). The mixture is warmed to 60° C. for 24 h then concentrated. A saturated solution of potassium carbonate (3 ml) is added and the solid is filtered, washed with cold water and dried under vacuum.

### Route D

To 1-N-substituted-barbiturate (0.5 mmol) is added an aldehyde (1.1 equivalent) in ethanolic solution. The mixture is warmed to 75° C. for 18 h and then concentrated. The resulting residue is diluted in methanol and solubilized after reflux. After cooling the solution, a solid precipitates which is collected by filtration and dried under vacuum.

### Route AC

To a suspension of a urea (2 mmol) in ethanol (2 ml) is added a 1 M ethanolic solution of sodium ethoxide (1.5 equivalent) and diethyl malonate (1.05 equivalent). The mixture is warmed to 60° C. for 18 h. After cooling the solution is concentrated under vacuum.

To the crude product is added an aldehyde (0.8 equivalent) in a solution of acetic acid. The mixture is warmed to 60° C. for 24 h then concentrated. A saturated solution of potassium carbonate (3 ml) is added and the solid is filtered, washed with cold water, and dried under vacuum.

### Route BD

To a urea (0.5 mmol) is added malonyl dichloride (1.1 equivalent) in solution in toluene. The mixture is warmed to 105° C. for 4 h and then concentrated under vacuum.

To the crude product is added an aldehyde (1.1 equivalent) in a solution in ethanol. The mixture is warmed to 75° C. for 18 h then concentrated. The residue obtained is diluted in methanol and solubilized after reflux. Upon cooling a solid precipitates from the solution, which solid is filtered and dried under vacuum.

In all these routes, compounds can be obtained as a mixture of isomers or as a single isomer. Compounds were often tested as a mixture of isomers.

The following Table 1 indicates compounds which have been synthesized, and also gives MS data, when available (N meaning that the value is not available), as well as the route of synthesis used.

TABLE 1

| Example | NAME | Formula | MS | Method |
|---|---|---|---|---|
| 1 | 5-(1H-Indol-3-ylmethylene)- naphthalen-1-yl-pyrimidine-2,4,6- trione | 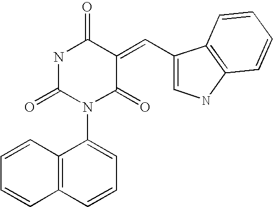 | 382 ES+ | A + C |
| 2 | 5-(1-Methyl-1H-indol-3-ylmethylene)- 1-naphthalen-1-yl-pyrimidine-2,4,6- trione | 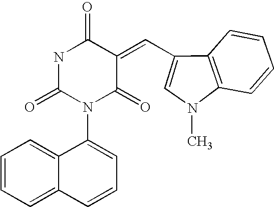 | 396 ES+ | A + C |
| 3 | 1-Benzyl-5-(1H-indol-3-yl methylene)-pyrimidine- 2,4,6-trione | 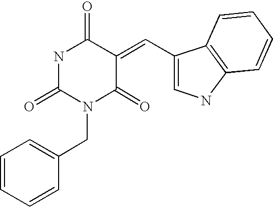 | 346 ES+ | AC |
| 4 | 1-(4-Methoxy-phenyl)-5-(1-methyl- 1H-indol-3-ylmethylene)-pyrimidine- 2,4,6-trione | 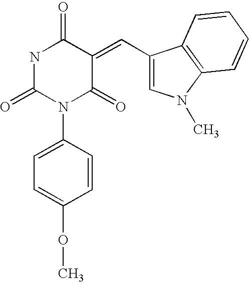 | 376 ES+ | AC |
| 5 | 1-(4-Chloro-phenyl)-5-(3- phenyl-allylidene)-pyrimidine- 2,4,6,-trione | 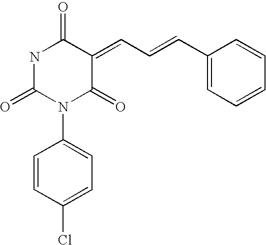 | 352 ES− | AC |
| 6 | 1-(4-Methoxy-phenyl)-5-(3-phenyl- allylidene)-pyrimidine-2,4,6-trione | 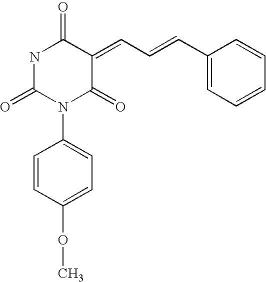 | 347 ES− | AC |
| 7 | 1-(4-Chloro-phenyl)-5-(1H-indol-3- ylmethylene)-pyrimidine- 2,4,6-trione | 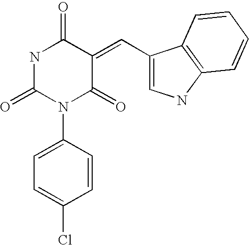 | 365 ES+ | AC |
| 8 | 1-Benzyl-5-(3-phenyl- allylidene)-pyrimidine-2,4,6-trione | 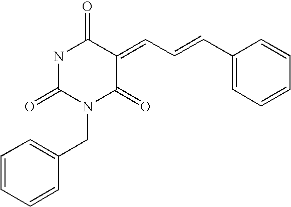 | 331 ES− | AC |
| 9 | 5-(1H-Indol-3-ylmethylene)-1-(4- methoxy-phenyl)-pyrimidine- 2,4,6-trione | 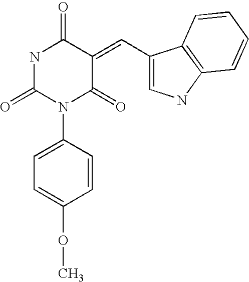 | 362 ES+ | AC |
| 10 | 1-(4-Chloro-phenyl)-5-(1-methyl-1H- indol-3-ylmethylene)-pyrimidine- 2,4,6-trione | 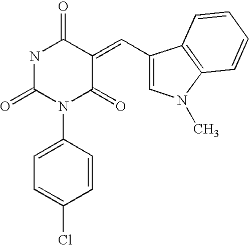 | 380 ES+ | AC |
| 11 | 1-Benzyl-5-(1-methyl-1H-indol-3- ylmethylene)-pyrimidine-2,4,6- trione | 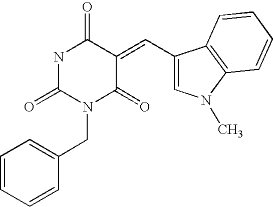 | 360 ES+ | AC |
| 12 | 1-Phenyl-5-(3-phenyl-allylidene)- pyrimidine-2,4,6-trione | 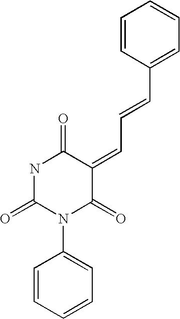 | 319 ES+ | AC |
| 13 | 5-(1H-Indol-3-ylmethylene)-1-phenyl- pyrimidine-2,4,6-trione | 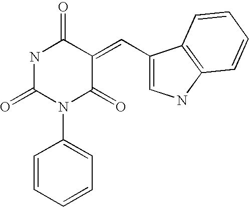 | 332 ES+ | AC |
| 14 | 5-(1-Methyl-1H-indol-3-ylmethylene)- 1-phenyl-pyrimidine-2,4,6-trione | 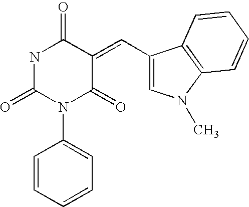 | 346 ES+ | AC |
| 15 | 5-(1-Methyl-1H-indol-3-ylmethylene)- 1-o-tolyl-pyrimidine-2,4,6-trione | 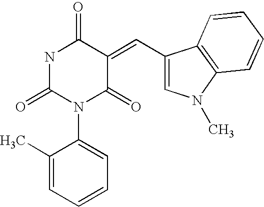 | 360 ES+ | AC |
| 16 | 5-(3-Phenyl-allylidene)-1-m-tolyl- pyrimidine-2,4,6-trione | 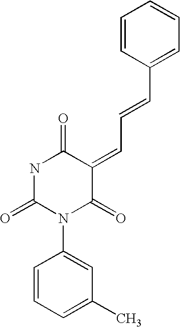 | 333 ES+ | AC |
| 17 | 5-(1H-Indol-3-ylmethylene)-1-m-tolyl- pyrimidine-2,4,6-trione | 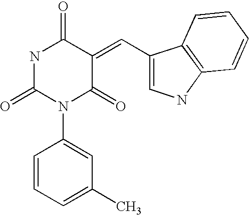 | 344 ES− | AC |
| 18 | 1-(2-Chloro-phenyl)-5-(1H-indol-3- ylmethylene)-pyrimidine-2,4,6- trione | 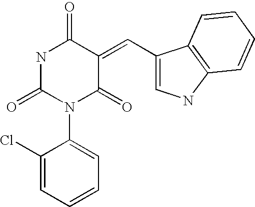 | 364 ES− | BD |
| 19 | 1-Phenyl-5-(thiophen-3- ylmethylene)-pyrimidine-2,4,6- trione | 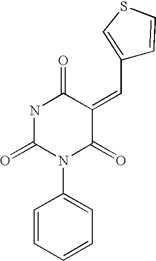 | 298 APCI- | AC |
| 20 | 5-(Pyrrol-2-ylmethylene)-1-phenyl- pyrimidine-2,4,6-trione | 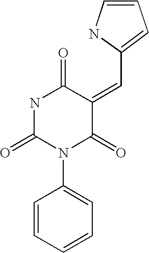 | 282 ES+ | AC |
| 21 | 5-(Benzofuran-2-ylmethylene)-1- phenyl-pyrimidine-2,4,6-trione | 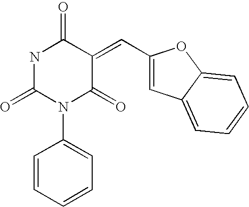 | 331 ES− | AC |
| 22 | 5-(Pyrrol-2-ylmethylene)-1-o-tolyl- pyrimidine-2,4,6-trione | 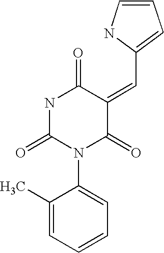 | 296 ES+ | AC |
| 23 | 5-(Thiophen-3-ylmethylene)-1-m- tolyl-pyrimidine-2,4,6-trione | 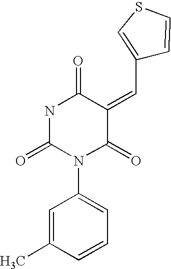 | 312 APCI− | AC |
| 24 | 5-(Pyrrol-2-ylmethylene)-1-m-tolyl- pyrimidine-2,4,6-trione | 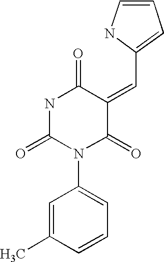 | 294 ES− | AC |
| 25 | 5-(Benzofuran-2-ylmethylene)-1-m- tolyl-pyrimidine-2,4,6-trione | 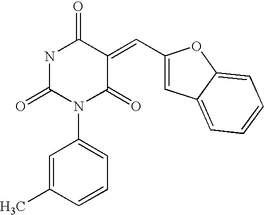 | 347 ES+ | AC |
| 26 | 1-(2-Chloro-phenyl)-5-(thiophen-3- ylmethylene)-pyrimidine-2,4,6- trione | 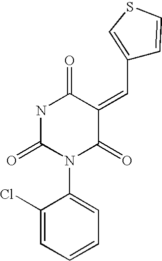 | 332 APCI− | AC |
| 27 | 1-(2-Chloro-phenyl)-5-(Pyrrol-2- ylmethylene)-pyrimidine-2,4,6- trione | 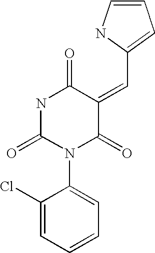 | 316 ES+ | AC |
| 28 | 5-(Benzofuran-2-ylmethylene)-1-(2- chloro-phenyl)-pyrimidine-2,4,6- trione | 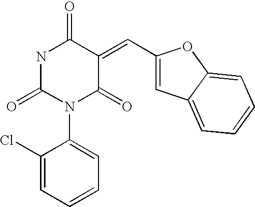 | 366 APCI− | AC |
| 29 | 1-Naphthalen-1-yl-5-(pyridin-4- ylmethylene)-pyrimidine-2,4,6- trione | 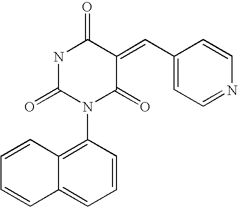 | 344 APCI+ | A + C |
| 30 | 5-[3-(4-Dimethylamino-phenyl)- allylidene]-1-naphthalen-1-yl- pyrimidine-2,4,6-trione | 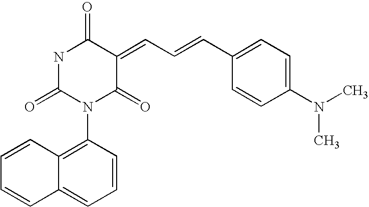 | 312 APCI+ | A + C |
| 31 | 5-[3-(4-Methoxy-phenyl)-allylidene]-1- naphthalen-1-yl-pyrimidine-2,4,6- trione | 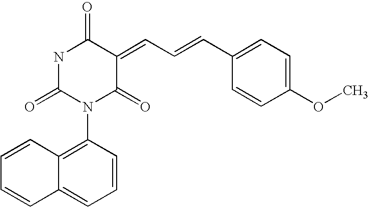 | 399 APCI+ | A + C |
| 32 | 5-(Benzofuran-2-ylmethylene)-1- naphthalen-1-yl-pyrimidine-2,4,6- trione | 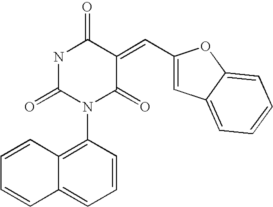 | 383 APCI+ | A + C |
| 33 | 1-Naphthalen-1-yl-5-(1H-pyrrol-2- ylmethylene)-pyrimidine-2,4,6- trione | 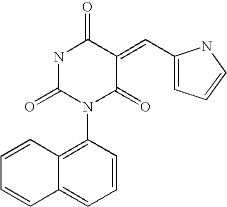 | 332 APCI+ | A + C |
| 34 | 1-Naphthalen-1-yl-5-(thiophen-3- ylmethylene)-pyrimidine-2,4,6- trione | 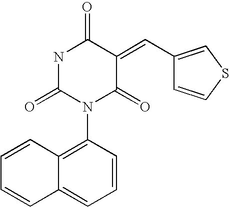 | 349 APCI+ | A + C |
| 35 | 5-(3-Phenyl-allylidene)-1-tolyl- pyrimidine-2,4,6-trione | 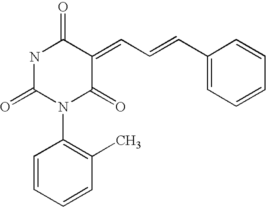 | 333 ES+ | B + D |
| 36 | 1-(2-Chloro-phenyl)-5-(1-methyl-1H- indol-3-ylmethylene)-pyrimidine- 2,4,6-trione | 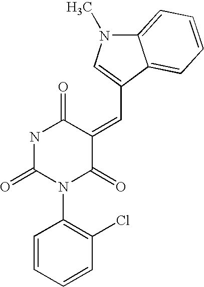 | 380 APCI+ | BD |
| 37 | 1-(3-Chloro-phenyl)-5-(1H-indol-3- ylmethylene)-pyrimidine-2,4,6- trione | 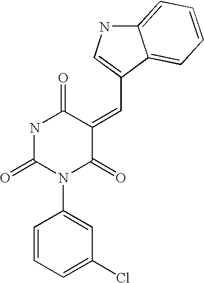 | 364 APCI− | BD |
| 38 | 1-(3-Chloro-phenyl)-5-(1-methyl-1H- indol-3-ylmethylene)-pyrimidine- 2,4,6-trione | 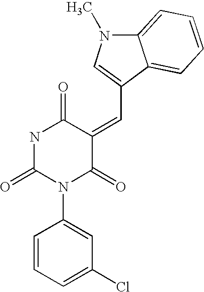 | 407 APCI− | BD |
| 39 | 5-(1-Methyl-1H-indol-3-ylmethylene)- 1-m-tolyl-pyrimidine-2,4,6-trione | 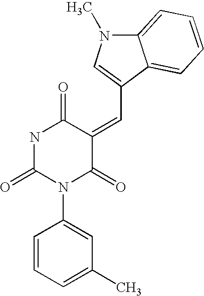 | 360 APCI+ | BD |
| 40 | 5-(1H-Indol-3-ylmethylene)-1-p-tolyl- pyrimidine-2,4,6-trione | 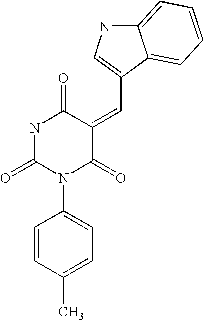 | 344 APCI− | BD |
| 41 | 5-(1-Methyl-1H-indol-3-ylmethylene)- 1-p-tolyl-pyrimidine-2,4,6-trione | 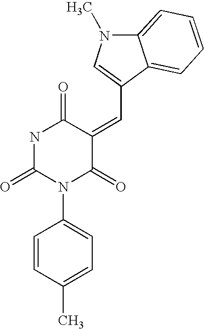 | 360 APCI+ | BD |
| 42 | 5-(3-Phenyl-allylidene)-1-p-tolyl- pyrimidine-2,4,6-trione | 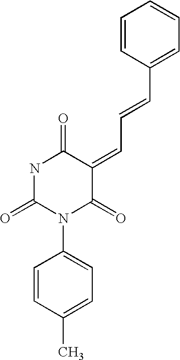 | 331 APCI− | BD |
| 43 | 1-(2-Chloro-phenyl)-5-(3-phenyl- allylidene)-pyrimidine-2,4,6-trione | 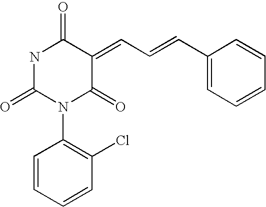 | 351 APCI− | BD |
| 44 | 1-1,3-Benzodioxol-5-yl-5-(3-phenyl- allylidene)-pyrimidine-2,4,6-trione | 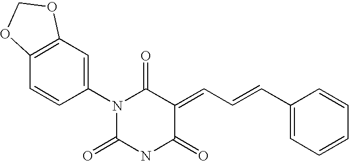 | 361 APCI− | A + C |
| 45 | 5-(1H-Indol-3-ylmethylene)-1-(3- methoxy-phenyl)-pyrimidine-2,4,6- trione | 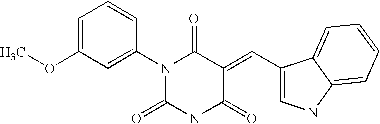 | 360 APCI− | A + C |
| 46 | 1-(2,3-Dihydro-1,4-benzodioxin-6-yl)- 5-(1H-indol-3-ylmethylene)- pyrimidine-2,4,6-trione | 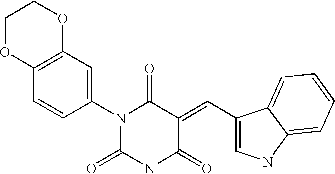 | 388 APCI− | A + C |
| 47 | {4-[2,4,6-Trioxo-5-(3-phenyl- allylidene)-tetrahydro-pyrimidin-1- yl]-phenyl}-acetic acid | 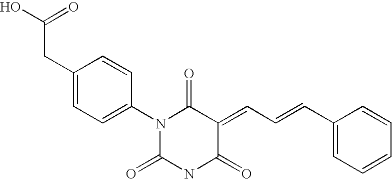 | 375 APCI− | A + C |
| 48 | {4-[5-(1H-lndol-3-ylmethylene)-2,4,6- trioxo-tetrahydro-pyrimidin-1-yl]- phenyl}-acetic acid | 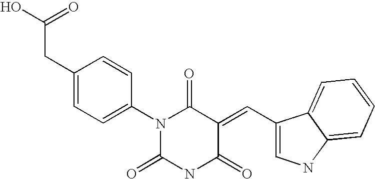 | 388 APCI− | A + C |
| 49 | 5-(1H-Indol-3-ylmethylene)-1-pyridin- 3-ylmethyl-pyrimidine-2,4,6-trione | 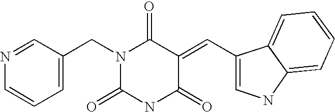 | 345 APCI− | A + C |
| 50 | 1-(1,3-Benzodioxol-5-yl)-5-(1H-indol-3- ylmethylene)-pyrimidine-2,4,6- trione | 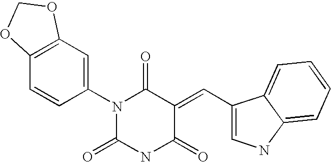 | 374 APCI− | A + C |
| 51 | 5-(3-Phenyl-allylidene)-1-quinolin-8- yl-pyrimidine-2,4,6-trione | 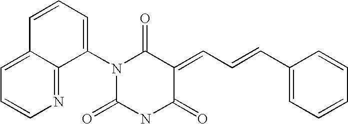 | 369 APCI− | A + C |
| 52 | 5-(1H-Indol-3-ylmethylene)-1- quinolin-8-yl-pyrimidine-2,4,6-trione | 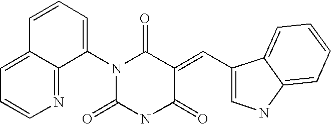 | 381 APCI− | A + C |
| 53 | 1-(3,4-Dimethoxy-phenyl)-5-(3- phenyl-allylidene)-pyrimidine-2,4,6- trione | 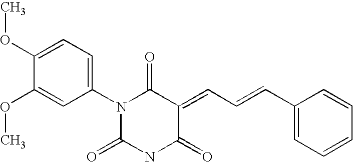 | 379 ES+ | A + C |
| 54 | 1-(3,4-Dimethoxy-phenyl)-5-(1H-indol- 3-ylmethylene)-pyrimidine-2,4,6- trione | 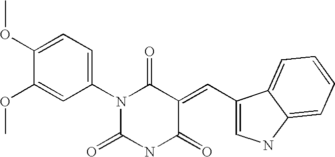 | 390 APCI− | A + C |
| 55 | 5-(3-Phenyl-allylidene)-1,3-di-m-tolyl- pyrimidine-2,4,6-trione | 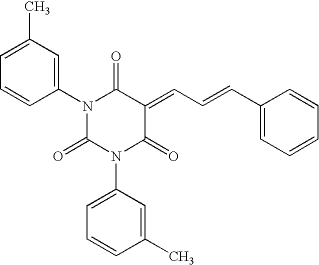 | 421 APCI− | B + D |
| 56 | 5-(1H-Indol-3-ylmethylene)-1,3-di-m- tolyl-pyrimidine-2,4,6-trione | 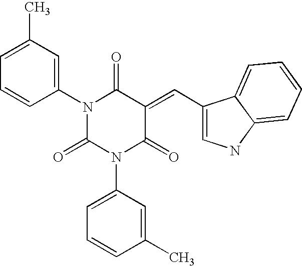 | 436 APCI+ | B + D |
| 57 | 3-[5-(1H-Indol-3-ylmethylene)-2,4,6- trioxo-tetrahydro-pyrimidin-1-yl]- benzonitrile | 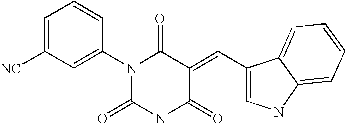 | 355 APCI− | A + D |
| 58 | {4-[5-(1H-Indol-3-ylmethylene)-2,4,6- trioxo-tetrahydro-pyrimidin-1-yl]- phenyl}-acetonitrile | 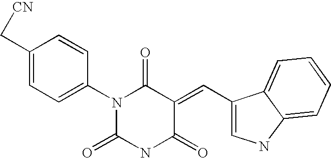 | 369 APCI− | A + D |
| 59 | 3-[2,4,6-Trioxo-5-{3-phenyl-allylidene)- tetrahydro-pyrimidin-1-yl]- benzonitrile | 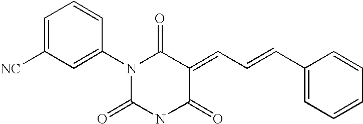 | 342 APCI− | A + D |
| 60 | 5-(Furan-2-ylmethylene)-pyrimidine- 2,4,6-trione | 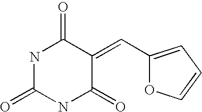 | 205 ES− | D |
| 61 | 5-(1H-Indol-3-ylmethylene)- pyrimidine-2,4,6-trione | 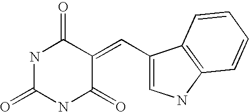 | 254 ES− | D |
| 62 | 5-(3-Phenyl-allylidene)-pyrimidine- 2,4,6-trione | 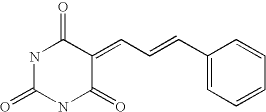 | 241 ES− | D |
| 63 | 1-Naphthalen-1-yl-5-(3-phenyl- allylidene)-pyrimidine-2,4,6-trione | 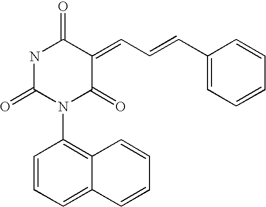 | 367 ES− | A + C |
| 64 | 5-(4-Dimethylamino-benzylidene)-1- quinolin-8-yl-pyrimidine-2,4,6-trione | 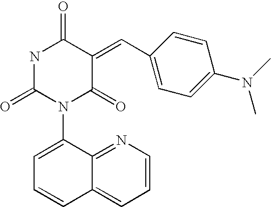 | 385 ES− | A + D |
| 65 | 5-(1-Methyl-1H-indol-3-ylmethylene)- 1-quinolin-8-yl-pyrimidine-2,4,6- trione | 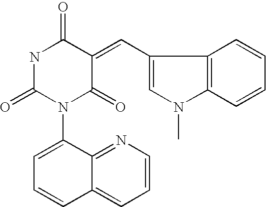 | 395 ES− | A + D |
| 66 | 5-Furan-2-ylmethylene-1-quinolin-8- yl-pyrimidine-2,4,6-trione | 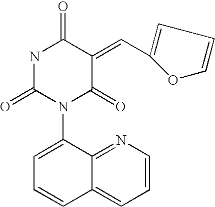 | 332 ES− | A + D |
| 67 | 5-(3-Phenyl-allylidene)-1-quinolin-5-yl- pyrimidine-2,4,6-trione | 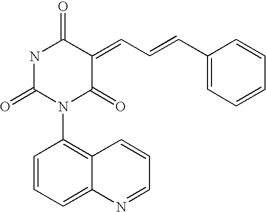 | 368 ES− | A + D |
| 68 | 1-Isoquinolin-5-yl-5-(3-phenyl- allylidene)-pyrimidine-2,4,6-trione | 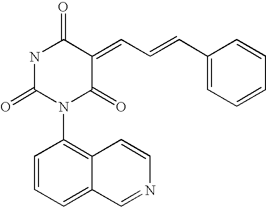 | 368 ES− | A + D |
| 69 | 5-(1H-Indol-3-ylmethylene)-1- isoquinolin-5-yl-pyrimidine-2,4,6- trione | 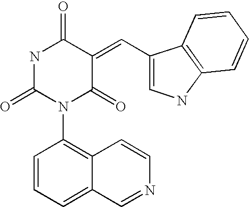 | 381 ES− | A + D |
| 70 | 5-(1H-Indol-3-ylmethylene)-1- quinolin-5-yl-pyrimidine-2,4,6- trione | 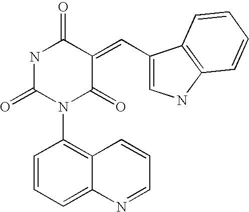 | 381 ES− | A + D |

Biological Results. Adhesion Inhibition Assay Method.

RPMI 8866 cells are used in this assay. They are available in a medium with 10% FBS, 200 mM L-Glutamine, and penicillin/streptomycin 10×.

1. Coat plates (costar 3590) with a human MAdCAM-1-Ig dilution prepared in carbonate buffer (25 ng of huMAdCAM/well in 50 μl carbonate buffer) overnight at 4° C. Carbonate buffer is NaHCO₃ 3.44 g, Na₂CO₃ 1.72 g, water qs 200 ml; adjust pH to 9.5, filter thru 0.2 μm and store at 4° C.

2. Wash plates with the plate washer (BioTeck Instrurnents EL404) once, using settings for washing cell rinse, with the following parameters:

| | |
|---|---|
| wash volume | 500 μl |
| wash cycle 1x | |
| soak time 0 | |
| wash depth | 80 (residual volume = 20 μl) |
| aspirate after wash | |
| shake time 0 | |
| washing buffer | HBSS, MnCl₂ 0.5 mM |

3. Block wells with blocking buffer (10% calf serum, PBS), 100 μl/well, 37° C., 3 h.

4. Wash plates with the plate washer once, using settings for washing cell rinse. Use plates within 10 min after washing.

5. Label the RPMI 8866 cells with 2′,7′-bis-(2-carboxyethyl)-5-6-carboxy-fluorescein acetoxymethyl ester (BCEF-AM) as follows:

spin down the cells at 1500 rpm, 10 min. Decant supernatant. Resuspend cells at 4×10⁶ cells/ml sterile PBS in 50 ml polypropylene tube. Add 2 μl BCEF-AM per ml of resuspended cells. Mix well and wrap tube in aluminum foil (to exclude light). Place cells at 37° C. for 30 min to label. Spin labeled cells down (while covered in aluminum foil) at 1500 rpm, 10 min. Decant supernatant. Resuspend labeled cells in 25 ml assay buffer and centrifuige at 1500 rpm, 10 min. Resuspend labeled cells in assay buffer at 2.5×10⁶ cells/ml. (assay buffer: HBSS, penicillin/streptomycin 10×, MnCl₂ 0.5 mM, Hepes 25 mM, pH=7.2)

6. Prepare dilutions of positive control: LDP-02 (α4β7 integrin blocking antibody) in assay buffer at 250 μg/ml and compounds of the invention in DMSO for addition to assay plates.

7. To polypropylene plate, add, with Multimek:

2.2 μl/well of 100% DMSO or compound dilutions;

147.8 μl/well of assay buffer to all wells;

50 μl/well of BCEF-labeled RPMI 8866 cells (at 2.5×10⁶ cells/ml assay buffer);

Mix well and add 180 μl of the mix to huMAdCAM-1-Ig coated plate.

8. Read plate in fluorescent plate reader (excite at 485 nm, read at 535 nm) and adjust the gain (total signal).

9. Wrap plates in aluminum foils and incubate at 25° C. for 30 min.

10. Wash plates with the plate washer using settings for washing cell rinse.

11. Read plate in florescent plate reader (excite at 485 nm, read at 535 nm).

The following Table 2 summarizes the results for some of the compounds and indicates the IC₅₀ in μM.

TABLE 2
α4β7 activity of preferred compounds

| Example | IC₅₀ (μM) |
|---|---|
| 1 | 0.41 |
| 16 | 0.9 |
| 2 | 0.94 |
| 49 | 1.3 |
| 43 | 1.9 |
| 15 | 2.3 |
| 18 | 2.5 |
| 42 | 2.6 |
| 35 | 2.65 |
| 51 | 2.7 |
| 37 | 2.75 |
| 17 | 2.93 |
| 24 | 2.95 |

### Equivalents

Those skilled in the art will recognize, or be able to ascertain using no more than routine experimentation, many equivalents to the specific embodiments and methods described herein. Such equivalents are intended to be encompassed by the scope of the following claims.

All patents, patent applications, and literature references cited herein are hereby expressly incorporated by reference.

1. A pharmaceutical composition comprising a compound of the following formula (I): 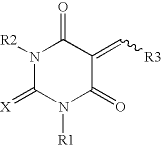
in which:
X is O or S;
R1 is H or —(CH₂)ₙ—Ar1, in which
n is 0, 1 or 2;
Ar1 is an aryl or heteroaryl group, optionally being substituted with one to three groups chosen independently from:
halogen,
cyano,
hydroxy,
carboxyl,
C₁-C₄ alkyl, optionally substituted with halo, cyano, hydroxy, carboxy,
C₁-C₄ haloalkyl,
C₁-C₄ alkoxy,
NR′R″ where R′ and R″ independently are hydrogen or a C₁-C₄ alkyl;
R2 is hydrogen or has independently the same meaning as R1;
R3 is Ar2 or —(CH═CH)—Ar2 where Ar2 has independently the same meaning as Ar1;
as well as the pharmaceutically acceptable derivatives thereof,
together with a pharmaceutically acceptable carrier.

2. The composition according to claim 1, in which in the formula (I):
R1 is —(CH₂)ₙ—Ar1, n and Ar1 being such as defined in claim 1
R2 has independently the same meaning as R1.

3. The composition according to claim 1, in which in the formula (I)
R1 is —(CH₂)ₙ—Ar1, n and Ar1 being such as defined in claim 1
R2 is hydrogen.

4. The composition according to claim 1, in which in the formula (I):
X is O;
R1 is —(CH₂)ₙ—Ar1, in which
n is 0 or 1;
Ar1 is an aryl or heteroaryl group, optionally being substituted with one or two groups chosen independently from halogen, C₁-C₄ alkyl and C₁-C₄ alkoxy;
R2 is hydrogen;
R3 is Ar2 or —(CH═CH)—Ar2 where Ar2 is an aryl or heteroaryl group, optionally being substituted with one C₁-C₄ alkyl group.

5. The composition according to claim 1, in which in the formula (I):
X is O;
R1 is selected from the following substituents: 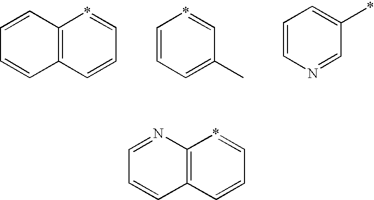
wherein the * indicates the site of binding where R1 is linked to the rest of (I);
R2 is hydrogen;
R3 is selected from the following substituents: 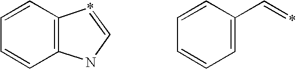
wherein the * indicates the site of binding where R1 is linked to the rest of (I).

6. The composition according to claim 1, in which the compound of formula (I) is selected from the group consisting of:

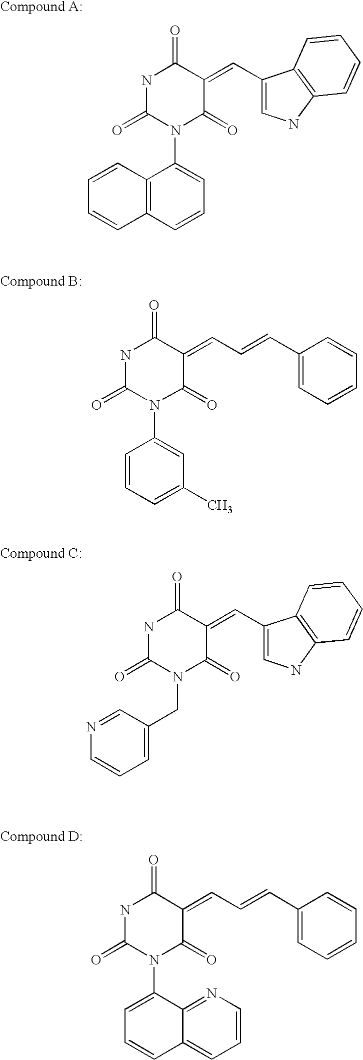

7. A method of treating an individual suffering from a disease associated with leukocyte infiltration of tissues expressing the molecule MAdCAM-1, comprising administering a therapeutically effective amount of an integrin antagonist of the following formula (I): 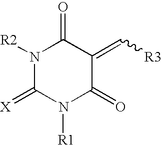
in which:
X is O or S;
R1 is hydrogen or —(CH₂)ₙ—Ar1, in which
n is 0, 1 or 2;
Ar1 is an aryl or heteroaryl group, optionally being substituted with one to three groups chosen independently from:
halogen,
cyno,
hydroxy,
carboxyl,
C₁-C₄ alkyl, optionally substituted with halo, cyano, hydroxy, carboxy,
C₁-C₄ haloalkyl,
C₁-C₄ alkoxy,
NR′R″ where R′ and R″ independently are hydrogen or a C₁-C₄ alkyl,
R2 is hydrogen or has independently the same meaning as R1;
R3 is Ar2 or —(CH═CH)—Ar2 where Ar2 has independently the same meaning as Ar1;
as well as the pharmaceutically acceptable derivatives thereof,
together with a pharmaceutically acceptable carrier.

8. The method according to claim 7, in which in the formula (I):
R1 is —(CH₂)ₙ—Ar1, n and Ar1 being such as defined in claim 7
R2 has independently the same meaning as R1.

9. The method according to claim 7, in which in the formula (I):
R1 is (CH₂)ₙ—Ar1, n and Ar1 being such as defined in claim 7
R2 is hydrogen.

10. The method according to claim 7, in which in the formula (I):
X is O;
R1 is CH₂)ₙ—Ar1, in which
n is 0 or 1;
Ar1 is an aryl or heteroaryl group, optionally being substituted with one or two groups chosen independently from halogen, C1-C4 alkyl and C1-C4 alkoxy;
R2 is hydrogen;
R3 is Ar2 or —(CH═CH)—Ar2 where Ar2 is an aryl or heteroaryl group, optionally being substituted with one C1-C4 alkyl group.

11. The method according to claim 7, in which in the formula (I):
X is O;
R1 is selected from the following substituents: 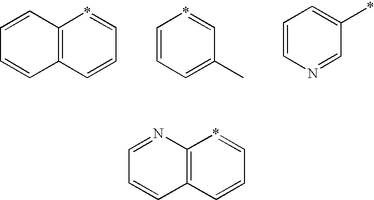
wherein the * indicates the site of binding where R1 is linked to the rest of (I);
R2 is hydrogen;
R3 is selected from the following substituents: 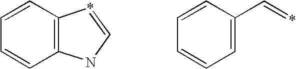
wherein the * indicates the site of binding where R1 is linked to the rest of (I).

12. The method according to claim 7, in which the compound of formula (I) is selected from the group consisting of:

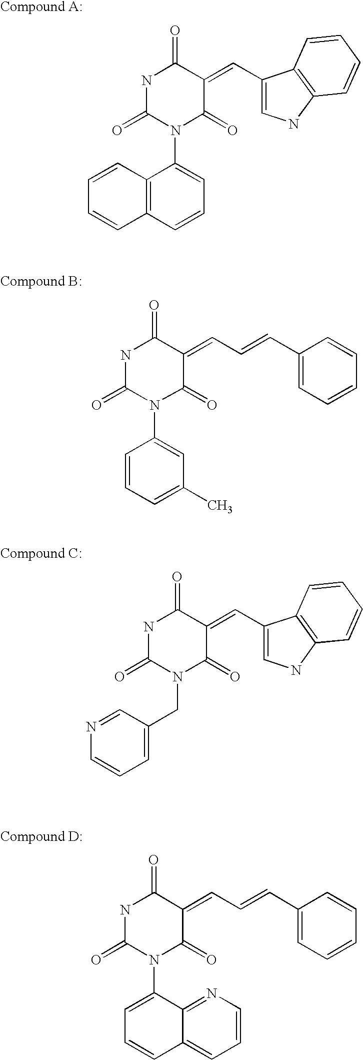

13. The method according to claim 7, in which the disease is an inflammatory disease.

14. The method according to claim 12, in which the disease is an inflammatory disease.

15. A method of inhibiting the binding of a cell expressing a ligand for α4β7 on the cell surface to MAdCAM-1 or a portion thereof, comprising contacting the cell with an effective amount of an integrin antagonist of the following formula (I): 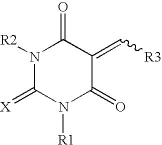
in which:
X is O or S;
R1 is hydrogen or —(CH₂)ₙ—Ar1, in which
n is 0, 1 or 2;
Ar1 is an aryl or heteroaryl group, optionally being substituted with one to three groups chosen independently from:
halogen,
cyano,
hydroxy,
carboxyl,
C₁-C₄ alkyl, optionally substituted with halo, cyano, hydroxy, carboxy,
C₁-C₄ haloalkyl,
C₁-C₄ alkoxy,
NR′R″ where R′ and R″ independently are hydrogen or a C₁-C₄ alkyl,
R2 is hydrogen or has independently the same meaning as R1;
R3 is Ar2 or —(CH═CH)—Ar2 where Ar2 has independently the same meaning as Ar1;
as well as the pharmaceutically acceptable derivatives thereof,
together with a pharmaceutically acceptable carrier.

16. The method according to claim 15, in which in the formula (I):
R1 is —(CH₂)ₙ—Ar1, n and Art being such as defined in claim 15
R2 has independently the same meaning as R1.

17. The method according to claim 15, in which in the formula (I):
R1 is —(CH₂)ₙ—Ar1, n and Art being such as defined in claim 15
R2 is hydrogen.

18. The method according to claim 15, in which in the formula (I):
X is O;
R1 is (CH₂)ₙ—Ar1, in which
n is 0 or 1;
Ar1 is an aryl or heteroaryl group, optionally being substituted with one or two groups chosen independently from halogen, C₁-C₄ alkyl and C₁-C₄ alkoxy;
R2 is hydrogen;
R3 is Ar2 or —(CH═CH)—Ar2 where Ar2 is an aryl or heteroaryl group, optionally being substituted with one C1-C₄ alkyl group.

19. The method according to claim 15, in which in the formula (I):
X is O;
R1 is selected from the following substituents: 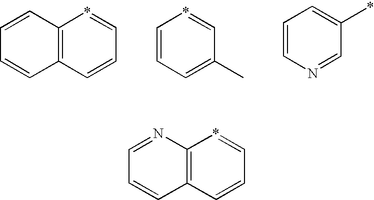
wherein the * indicates the site of binding where R1 is linked to the rest of (I);
R2 is hydrogen;
R3 is selected from the following substituents: 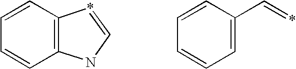
wherein the * indicates the site of binding where R 1 is linked to the rest of (I).

20. The method according to claim 15, in which the compound of formula (I) is selected from the group consisting of:

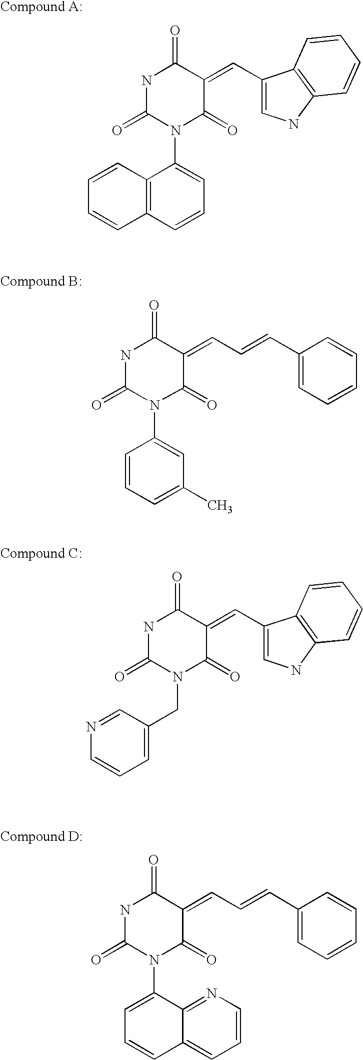

21. The method according to claim 15, in which the ligand is human α4β7 integrin.

22. The method according to claim 15, in which the cell is a leukocyte.

23. The method according to claim 15, in which MAdCAM-1 is expressed on the surface of an endothelial cell.

24. A method for preparing a pharmaceutical composition comprising a compound of the following formula (I): 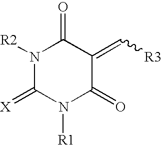
in which:
X is O or S;
R1 is hydrogen or —(CH₂)ₙ—Ar1, in which
n is 0, 1 or 2;
Ar1 is an aryl or heteroaryl group, optionally being substituted with one to three groups chosen independently from:
halogen,
cyano,
hydroxy,
carboxyl,
C₁-C₄ alkyl, optionally substituted with halo, cyano, hydroxy, carboxy,
C₁-C₄ haloalkyl,
C₁-C₄ alkoxy,
NR′R″ where R′ and R″ independently are hydrogen or a C₁-C₄ alkyl,
R2 is hydrogen or has independently the same meaning as R1;
R3 is Ar2 or —(CH═CH)—Ar2 where Ar2 has independently the same meaning as Ar1;
as well as the pharmaceutically acceptable derivatives thereof,
together with a pharmaceutically acceptable carrier;
said process comprising the steps of:
(i) reacting a compound: 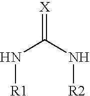
with diethyl malonate or malonyl dichloride to yield the following compound: 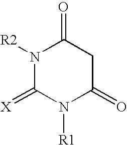
(ii) reacting said compound with an aldehyde of formula R3-CHO to yield the compound of formula I; and
(iii) mixing said compound with the pharmaceutically acceptable carrier.

25. The method according to claim 24, in which in the formula (I)
R1 is —(CH₂)ₙ—Ar1, n and Ar1 being such as defined in claim 24
R2 has independently the same meaning as R1.

26. The method according to claim 24, in which in the formula (I):
R1 is —(CH₂)ₙ—Ar1, n and Ar1 being such as defined in claim 24
R2 is hydrogen.
